SEQUENCE LISTING (1 sequence)

SEQ ID NO: 1 - DNA, 3122 bp, mammalian
tcctagactggacagtgggcaaagtcctcccagcccccagggtcctggctcccttccctgtagcctgcttctggctgacaacagaagcagggccccaaggttaggcaaacaagctagtgataaggcacttccaggttgggccttgcattcaaggcccacccagctctggggctggcttcctggcttagcaaaagccctagtcttttgtgcacacaagagcgggcaccaatggggacacctgctgattgtgcacctggggccttggtgccctggtacagcctgagttaatgaccttgtttatccacttgagtcatctgataaggggcagctgagtgagcggcaggtggccctgtgccctgcaccggccacttcattgactgaggtgatatcagtgccacgtggggttcccaatgccccctcccccaccacttccccaccattcctgccaggggcaatgtctgtgtgtttttttcaatgaacatgacttctggagtcaaggttgttgggccattccccccgttccactcactgggaatataaatagcacccacagcgcagaacacagagccagagagctggaagtgagagcagatccctaaccatgagcaccagccaaccaggggcctgcccatgccagggagctgcaagccgccccgccattctctacgcacttctgagctccagcctcaaggctgtcccccgaccccgtagccgctgcctatgtaggcagcaccggcccgtccagctatgtgcacctcatcgcacctgccgggaggccttggatgttctggccaagacagtggccttcctcaggaacctgccatccttctggcagctgcctccccaggaccagcggcggctgctgcagggttgctggggccccctcttcctgcttgggttggcccaagatgctgtgacctttgaggtggctgaggccccggtgcccagcatactcaagaagattctgctggaggagcccagcagcagtggaggcagtggccaactgccagacagaccccagccctccctggctgcggtgcagtggcttcaatgctgtctggagtccttctggagcctggagcttagccccaaggaatatgcctgcctgaaagggaccatcctcttcaaccccggtgagctcccagacactcctgggtggggggcaaggctgaagcaggggccagccagggaaggtagcttctaagggtttgatatctgttatctcattgggtcctcattttctttttatttttctgagacagagtctcactctgtcacccagtctggagtgcagtggtgcgatcttggctcactgcaacctctgcctcctgggttcaagcaattttctctgcctcagcctcccgagtagctgggattacagatggccgccaccactcccggctaatttttgtactttttagtagagatgggctttcaccatgttgatcaagctggtcttgaactcctaacccctcaggtgatccacccaccttggcctcccaaagtgctgggattacaggcgtgagccaccgtgcttggccagtcctcattttcatcaaatagccatctcaccagaagaggcacagagaagcctaagtgactagctaaaggtaacacagccaggtagggacagaccaggatggcaactcaggtctattcactccagagactggcccttgccactctgcctgagaaatttgctctgacctttacagaaggctgtggggaggtggggtgggcagacacagctggtctcagtcacatccctcaaaggaaggctgcatggtgaagctctagagctaaaactccctctgtgggttcagaaagatcacatagaagagatgactgaaaggggcgtagggaggacaggaaaggagtctttatggaattcggccaggagagcggcagatggagacagtgctgtttgtaatctggagagcatctgagatgagagttccccaggagtcccttgtggcacgcataaatcgtagtaattaattgtaataatagtagcaaatactcatatcacacttaccatatgttaggccctattctaagggttttacatatactagttcatttaaatctcataaaagcctaggagacaggtcctattaatattattattcccgttttacagataaagaaactgaagcccagagattaagtgacttgcccaaggtcacccagctaataagtgacagtgctgggattcatacccaggaagcctaattcttaaccattactccacactgcctcttcataaatggatggatgaatacattgaaaattgataaataaattacctcctctaaaggaggaggtagtagtgggacctcaaaggccgagcaaaggaggcagaggggtgtctctgatggcccagatcttgggccagtcttgtcctttggtggctgggagtagggtgctcaccagccctcttctccctctctgcccacagatgtgccaggcctccaagccgcctcccacattgggcacctgcagcaggaggctcactgggtgctgtgtgaagtcctggaaccctggtgcccagcagcccaaggccgcctgacccgtgtcctcctcacggcctccaccctcaagtccattccgaccagcctgcttggggacctcttctttcgccctatcattggagatgttgacatcgctggccttcttggggacatgcttttgctcaggtgacctgttccagcccaggcagagatcaggtgggcagaggctggcagtgctgattcagcctggccatccccagaggtgacccaatgctcctggaggggcaagcctgtatagacagcacttggctccttaggaacagctcttcactcagccacaccccacattggacttccttggtttggacacagtgctccagctgcctgggaggcttttggtggtccccacagcctctgggccaagactcctgtcccttcttgggatgagaatgaaagcttaggctgcttattggaccagaagtcctatcgactttatacagaactgaattaagttattgatttttgtaataaaaggtatgaaacacttggagcctgatactgtcattttgtgcatatggaagggactgtctcacctccagggcccaccctgct

(19) United States
(12) Patent Application Publication
(10) Pub. No.: US 2009/0137516 A1
(43) Pub. Date: May 28, 2009

(54) COMPOSITIONS AND METHODS OF TREATING DYSLIPIDEMIA

(75) Inventors: Helen Bayes Hartman, Havertown, PA (US); Mark James Evans, Radnor, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/263,591

(22) Filed: Nov. 3, 2008

Related U.S. Application Data
(63) Continuation of application No. 60/985,482, filed on Nov. 5, 2007.

(51) Int. Cl. A61K 31/7088; A61P 9/10

(52) U.S. Cl. 514/44

(57) ABSTRACT

The invention relates to the treatment of subjects for the purpose of reducing serum LDL, VLDL, triglycerides and fatty acids, by administering agents which reduce the activity of the bile acid pathway component SHP. Methods and pharmaceutical preparations comprising such agents are provided.

This application claims benefit of priority of U.S. Application Ser. No. 60/985,482, filed Nov. 5, 2007, the disclosure of which is incorporated herein by reference in its entirety.

### FIELD

The invention is directed to medical therapies and therapeutics for treating dyslipidemia. More particularly, it is directed to compositions and methods useful in the reduction of serum low density lipoproteins, very low density lipoproteins, triglycerides and fatty acids, especially in subjects suffering from hypercholesterolemia or hypothyroidism.

### BACKGROUND

Cholesterol metabolism impacts many systems in the body and is an important health consideration. Cholesterol affects digestion, liver and gall bladder function, cell membrane integrity, steroid hormone metabolism, and vascular health. Given the importance of lowering serum cholesterol, namely cholesterol associated with low density lipoprotein (“LDL”), to prevent or reverse cardiovascular disease, a number of drugs have been developed to address this issue. Those drugs fall into two general classes: (1) drugs that slow the absorption of exogenous cholesterol through the intestine (e.g., phytosterols), and (2) drugs that inhibit endogenous production of cholesterol and concomitantly increases endogenous expression of LDL receptor (“LDL-R”) (e.g., statins). HMG-CoA (3-hydroxy-3-methylglutaryl-coenzyme A) reductase is involved in the endogenous synthesis of cholesterol and is a target of statins (see Marc Issandou, Pharmacology and Therapeutics, 111(2): 424-433, 2006) whereas ABCG5/8 (adenosine triphosphate binding cassette transporter genes 5 and 8) are involved in transport of sterols across the intestine (see Sudhop et al., Pharmacology and Therapeutics, 105(3): 333-341, 2005.) In addition to the synthesis and absorption of cholesterol, as it relates to serum cholesterol levels, cholesterol is removed from the system through the liver in association with bile acids as a component of bile.

Cholesterol metabolism is closely linked to the flux of bile acids through the hepatic and digestive system. When cholesterol levels increase in the liver, (1) oxysterols accumulate and activate liver X receptor (“LXR”), which is a member of the nuclear receptor superfamily, which in turn (2) stimulates the transcription of CYP7A1, which (3) results in increased bile acid (BA) synthesis and the subsequent excretion of cholesterol. As BA levels rise, (4) BA binds to its receptor, farnesoid X receptor (“FXR”), which in turn (5) induces expression of small heterodimer partner (“SHP”), which in turn (6) inhibits liver receptor homolog-1 (“LRH-1”) as well as LXR. Since LRH-1 is essential for CYP7A1 expression, the induction of SHP results in decreased CYP7A1 expression and concomitant decrease in BA production. For a review of the LXR-SHP system, see Trauner, M, Hepatology, 46(1):1-5, 2007, and Wang et al., Journal of Biological Chemistry, 278(45):44475-44481, 2003.

### SUMMARY

In one aspect, the invention provides methods for reducing the level of very low density lipoprotein (“VLDL”) or low density lipoprotein (“LDL”) in serum of a subject, the methods comprising inhibiting the activity of small heterodimer partner (“SHP”) in the subject.

In some embodiments, the invention provides methods for reducing VLDL or LDL in serum of a subject as described herein wherein the subject has familial hypercholesterolemia.

In some embodiments, the invention provides methods for reducing VLDL or LDL in serum of a subject as described herein wherein the subject has hypothyroidism.

In some embodiments, the invention provides methods for reducing VLDL or LDL in serum of a subject as described herein wherein the subject has diet-induced dyslipidemia.

In some embodiments, the invention provides methods for reducing VLDL or LDL in serum of a subject as described herein wherein the inhibition of SHP activity is accompanied by an increase in expression of FGF 15 in the ileum of the subject.

In some embodiments, the invention provides methods for reducing VLDL or LDL in serum of a subject as described herein wherein the inhibition of SHP activity is followed by a decrease in the level of serum triglycerides or fatty acids in the subject.

In some embodiments, the invention provides methods for reducing VLDL or LDL in serum of a subject as described herein wherein the patient is a human.

In some embodiments, the invention provides methods for reducing VLDL or LDL in serum of a subject as described herein wherein the step of inhibiting the activity of SHP comprises administering a therapeutically effective amount of a polynucleotide to the subject. In some embodiments, the polynucleotide is capable of causing a reduction in the amount of SHP produced in the liver. In some embodiments, the polynucleotide is an antisense polynucleotide.

In some embodiments, the antisense polynucleotide has the sequence that hybridizes under physiological conditions to all or part of a polynucleotide sequence that encodes SHP. In some embodiments, the polynucleotide is an siRNA molecule. In some embodiments, the polynucleotide is an shRNA molecule.

In some embodiments, the invention provides methods of inhibiting SHP activity as described herein, which comprise the selective degradation of the SHP messenger RNA produced in the liver.

In some embodiments, the invention provides methods of inhibiting SHP activity as described herein, which comprise the inhibition of translation of the SHP messenger RNA in the liver.

In some embodiments, the invention provides methods of inhibiting SHP activity as described herein, wherein the step of inhibiting the activity of SHP comprises administering a therapeutically effective amount of a compound to the subject, wherein the compound that is delivered to the subject is capable of being metabolized in the liver of the subject into an inhibitor of SHP.

In some embodiments, the invention provides methods of inhibiting SHP activity as described herein, wherein the step of inhibiting the activity of SHP comprises administering a therapeutically effective amount of a composition to the subject, wherein the composition is capable of inhibiting SHP activity or inhibiting SHP production. In some embodiments, the composition comprises any one or more of an antibody, an antibody fragment, an aptamer, an SHP fragment, an SHP analog or non-functional mimic thereof, an SHP target, target fragment or target mimic.

In some embodiments, the invention provides methods of inhibiting SHP activity as described herein wherein the step of inhibiting the activity of SHP comprises administering a therapeutically effective amount of a compound that is capable of being metabolized into an FXR antagonist in the liver.

In some embodiments, the invention provides methods of inhibiting SHP activity as described herein wherein the step of inhibiting the activity of SHP comprises administering a therapeutically effective amount of a compound that is capable of being metabolized into an estrogen receptor (“ER”) antagonist in the liver.

In another aspect, the invention provides compositions useful for the treatment of hyperlipidemia, wherein hyperlipidemia includes any one or more of the following conditions: elevated serum LDL, elevated serum VLDL, elevated serum triglycerides and elevated serum fatty acids.

In another aspect, the invention provides a medicament useful in the treatment of hyperlipidemia in a subject, the medicament comprising an inhibitor of SHP.

### BRIEF DESCRIPTION OF THE DRAWINGS

The following figures form part of the present specification and are included to further demonstrate certain aspects of the present invention. The invention may be better understood by reference to one or more of these figures in combination with the detailed description of specific embodiments presented herein.

FIG. 1 depicts gene expression in C57BI/6 WT mice, C57BI/6 FXR−/− mice, 129 WT mice, 129 SHP−/− mice.

FIG. 2 depicts lipid levels and inflammatory markers in C57BI/6 WT mice, C57BI/6 FXR−/− mice, 129 WT mice, 129 SHP−/− mice.

FIG. 3 depicts the protection of SHP−/− mice from diet-induced hypercholesterolemia.

FIG. 4 depicts the protection of hypothyroid SHP−/− mice from an increase LDL cholesterol levels.

FIG. 5 depicts the protection of LDLR−/−SHP−/− mice from Western diet dyslipidemia.

FIG. 6 depicts the protection against diet-induced dyslipidemia in mice by the selective loss of SHP expression in hepatocytes.

### DETAILED DESCRIPTION OF AN EMBODIMENT

It will be readily apparent to one skilled in the art that various embodiments and modifications may be made to the invention disclosed in this application without departing from the scope and spirit of the invention.

As used in the specification, “a” or “an” may mean one or more. As used in the claim(s), when used in conjunction with the word “comprising”, the words “a” or “an” may mean one or more than one.

The inventors have made the surprising discovery that by reducing or inhibiting the activity of the small heterodimer partner (“SHP”), serum levels of LDL, VLDL, triglycerides and their associated fatty acids are reduced in subjects having dyslipidemia, wherein dyslipidemia includes elevated serum levels of LDL, VLDL, triglycerides and their associated fatty acids. Dyslipidemia in the subject may be due to (1) a genetic defect(s) that impairs LDL homeostasis, such as e.g., LDL-R loss-of-function and familial hypercholesterolemia, (2) diet, and/or (3) hypothyroidism.

Thus, the invention is directed to methods for treating hyperlipidemia via blocking or otherwise reducing the activity of SHP in the liver of a subject. Hyperlipidemia includes elevated serum levels of VLDL, LDL, triglycerides and/or fatty acids relative to serum levels considered normal for the subject cohort. In some embodiments, the activity of SHP is reduced by at least 10% relative to the levels of activity in an untreated subject. In other embodiments the activity of SHP is reduced by at least 50%. The activity of SHP can be reduced or inhibited by any one or more of myriad means, such as by (1) genetically modifying the subject liver genome to eliminate one or both of the alleles encoding SHP, or to disrupt the cis-regulator sequences associated with one or both alleles of SHP; (2) stimulating inhibitors of SHP; (3) inhibiting stimulators of SHP; (4) blocking interaction of SHP with its downstream effectors; and/or inter alia (5) reducing the production of SHP protein.

In one embodiment, the invention is directed to lowering serum levels of LDL and VLDL in a subject suffering from diet-induced hypercholesterolemia, by reducing the production of SHP protein by the subject. Similarly, the invention is directed to lowering serum levels of LDL, VLDL, triglycerides and/or fatty acids in a subject suffering from familial hypercholesterolemia or hypothyroidism, by reducing the production of SHP protein by the subject. The production of SHP protein can be reduced in the subject by administering an effective amount of an antisense polynucleotide to the subject. In some embodiments, the antisense polynucleotide has a sequence that is capable of hybridizing to a portion of an SHP sense sequence, such as for example SEQ ID NO:1; GENBANK accession number of AB058644 (see e.g. Nishiqori et al., PNAS USA, 98(2):575-580, 2001, which is incorporated herein by reference). In some embodiments, the antisense polynucleotide is in a form that can reach the liver of the subject, such as for example as part of a vector that can target the liver. Adenovirus may be used as a vector to deliver the antisense polynucleotide to an hepatocyte in the subject. The antisense oligonucleotide may be delivered to the subject by way of any one or more of myriad means, such as for example viral delivery vectors, liposomes, chitosan-based vectors, nanoparticles, and the like (reviewed in Akhtar and Benter, Advanced Drug Delivery Reviews, 59(2,3):164-182, 2007, which is incorporated herein by reference).

In another aspect of this embodiment, the production of SHP protein can be reduced in the subject by administering an effective amount of an shRNA (small hairpin RNA) or an siRNA (small interfering RNA) polynucleotide to the subject. In general, shRNAs are processed by the cellular machinery into siRNAs. In some embodiments, the shRNA or siRNA polynucleotide has a sequence that is capable of targeting the SHP transcript for degradation, thereby reducing or preventing the production of SHP protein. In some embodiments, shRNAs comprise about 30 nucleotides that are capable of hybridizing to a portion of SEQ ID NO:1. In some embodiments, the shRNA is capable of being cleaved by the cellular machinery into an siRNA, which is capable of binding to an RNA-indiced silencing complex (RISC). This complex binds to and cleaves SHP mRNA. In some embodiments, siRNA comprises about 20 nucleotides that are capable of hybridizing to a portion of an SHP sense sequence, such as provided by way of example in SEQ ID NO:1. A target sequence for this siRNA can be selected by applying principles that are well known in the art. See for example Wadhwa et al., Mutation Research, 567:71-84, 2004, which is herein incorporated by reference. The siRNA may be delivered to the subject by way of one or more of myriad means, such as for example viral delivery vectors, iposomes, chitosan-based vectors, nanoparticles, and the like (reviewed in Akhtar and Benter, Advanced Drug Delivery Reviews, 59(2,3):164-182, 2007, which is incorporated herein by reference).

Methods for determining effective shRNAs for use in this invention are known in the art. Some of these methods are taught in whole or in part in McIntyre and Fanning, BMC Biotechnol. 6: 1, 2006; Harper et al., Proc. Natl. Acad. Sci. U.S.A. 102 (16): 5820-5, 2005; Nielsen et al., Retrovirology 2: 10, 2005; Paddison et al., Genes Dev. 16 (8): 948-58, 2002; and Cao et al., J. Appl. Genet. 46 (2): 217-25, 2005; which are herein incorporated by reference. Methods for determining effective siRNAs for use in this invention are also known in the art. Some of these methods are taught in whole or in part in Birmingham et al., Nat. Protoc., 2(9):2068-78, 2007; Krueger et al., Oligonucleotides, 17(2): 237-250, 2007; McQuisten and Peek, BMC Bioinformatics, 8: 184, 2007; Li and Cha, Cell. Mol. Life. Sci., 64(14): 1785-92, 2007; Amarzguioui et al., Nat. Protoc., 1(2): 508-17, 2006; Patzel V., Drug Discovery Today, 12(3-4): 139-48, 2007; Iyer et al., Comput. Methods Programs Biomed., 85(3):203-9, 2007; Ladunga I., Nucleic Acids Res., 35(2):433-40, 2007; Vert et al., BMC Bioinformatics, 7: 520, 2006; Gong et al., BMC Bioinformatics, 7: 516, 2006; Inoue et al., J. Drug Target, 14(7): 448-55, 2006; and Kurreck J., J. Biomed. Biotechnol., 2006(4): 83757, which are herein incorporated by reference. Also, several on-line programs may be employed to pick siRNA sequences for use in this invention, including for example the RNAi CODEX™ method (http://codex.cshl.edu/scripts/newmain.pl), Dharmacon's siDESIGN® Center (www.dharmacon.com/DesignCenter/DesignCenterPage.aspx), and Invitrogen's BLOCK-iT™ RNAi Designer (rnaidesigner.invitrogen.com/-rnaiexpress/index.jsp).

In another embodiment, the invention is directed to lowering serum levels of LDL and VLDL in a subject suffering from diet-induced hypercholesterolemia, or lowering serum levels of LDL, VLDL, triglycerides and/or fatty acids in a subject suffering from familial hypercholesterolemia or hypothyroidism, by administering to the subject a therapeutically effective amount of a compound in a pharmaceutically acceptable excipient, wherein the compound directly inhibits the activity of SHP at least in the liver of the subject. In an alternative aspect, the compound that is delivered to the subject is capable of being metabolized in the liver to form a second compound, which is capable of directly inhibiting the activity of SHP in the subject. An exemplary SHP inhibitor can include a competitive inhibitor that competes with a physiologically relevant receptor or binding partner of SHP, such as for example HDAC-1, G9a, histone 3 and fragments and/or analogs thereof. See Boulias and Talianidis, Nucleic Acids Research, 32(20):6096-6103, 2004, which describes the native receptors of SHP. Compounds that directly inhibit SHP also include antibodies, antibody fragments, SMIPs, ScFv polypeptides, aptamers, ligands, receptors, chaparones, synthetic biomolecules and the like, which are capable of binding to SHP and preventing it from reaching its native target in the cell.

In another embodiment, the invention is directed to lowering serum levels of LDL and VLDL in a subject suffering from diet-induced hypercholesterolemia, or lowering serum levels of LDL, VLDL, triglycerides and/or fatty acids in a subject suffering from familial hypercholesterolemia or hypothyroidism, by administering to the subject a therapeutically effective amount of a compound in a pharmaceutically acceptable excipient, wherein the compound inhibits the activity of FXR, which is an upstream effector of SHP, at least in the liver of the subject. In an alternative aspect, the compound that is delivered to the subject is capable of being metabolized in the liver to form a second compound, which is capable of inhibiting the activity of FXR in the subject. An inhibitor of FXR can be an antagonist of FXR. Non-limiting examples of FXR antagonists include phytosterols such as stigmasterol (Carter et al., Pediatric Research, 62(3):301-306, 2007), substituted isoxazole derivatives (Kainuma et al., Bioorg. Med. Chem., 15(7):2587-2600, 2007), 5-a-bile alcohols (Nishimaki-Mogami et al., Biochem. Biophys. Res. Comm., 339(1):386-391, 2005), and the hypolipidemic agent guggulsterone (Deng et al., J. Pharmacol. Exp. Ther., 320(3):1153-1162, 2006).

In another embodiment, the invention is directed to lowering serum levels of LDL and VLDL in a subject suffering from diet-induced hypercholesterolemia, or lowering serum levels of LDL, VLDL, triglycerides and/or fatty acids in a subject suffering from familial hypercholesterolemia or hypothyroidism, by administering to the subject a therapeutically effective amount of a compound in a pharmaceutically acceptable excipient, wherein the compound inhibits the activity of estrogen receptor (“ER”), which is an upstream effector of SHP, at least in the liver of the subject. In an alternative aspect, the compound that is delivered to the subject is capable of being metabolized in the liver to form a second compound, which is capable of inhibiting the activity of ER in the subject. An inhibitor of ER can be an antagonist of ER. A non-limiting example of an ER antagonist is EM-652-HCl and its prodrug EM-800 (Picard et al., International Journal of Obesity Related Metabolic disorders, 24(7):830-840, 2000).

In yet another embodiment, the invention provides compositions that modulate the activity of SHP in a subject, wherein the modulation reduces the level of LDL, VLDL, triglyceride and/or fatty acid in serum of the subject. In some embodiments, the compositions comprise compounds that inhibit the activity of SHP in a hepatocyte, such as for example, (1) by mimicking physiological targets of SHP and therefore competitively binding to SHP and reducing SHP's ability to find a physiological target, (2) by mimicking a binding site on SHP and therefore reducing the number of available targets for SHP to bind, and (3) by binding to SHP, and therefore blocking any sites accessible to the target(s). In some embodiments, the first aspect includes analogs and/or fragments of targets such as for example histone 3, histone 3 fragment, histone 3 analog, HDAC-1, HDAC-1 fragment, HDAC-1 analog, G9a, G9a fragment, and G9a analog. In some embodiments, the second aspect includes fragments or analogs of SHP that lack the full functionality of SHP. In some embodiments, the third instance includes, e.g., antibodies and their fragments, and aptamers and their fragments.

In some embodiments, the compositions comprise compounds that inhibit the production or expression of SHP in a hepatocyte, such as for example, (1) by inhibiting the transcription or translation of SHP, or (2) by targeting the SHP transcript for degradation. In some embodiments, the first aspect includes agents such as antisense polynucleotides that can hybridize to one or more regions of SEQ ID NO:1, and drugs that affect translation of SHP by affecting transcription initiation or translation initiation. In some embodiments, the second aspect includes siRNA molecules.

### EXAMPLE

The following example is included to demonstrate particular embodiments of the invention. It should be appreciated by those of skill in the art that the techniques disclosed in the example which follows represents techniques discovered by the inventor to function well in the practice of the invention, and thus can be considered to constitute modes for its practice. However, those of skill in the art should, in light of the present disclosure, appreciate that many changes can be made in the specific embodiments which are disclosed and still obtain a like or similar result without departing from the spirit and scope of the invention.

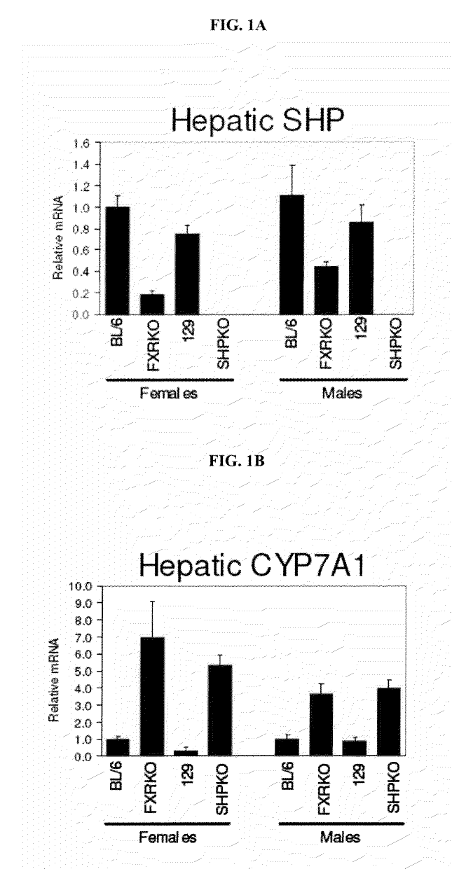

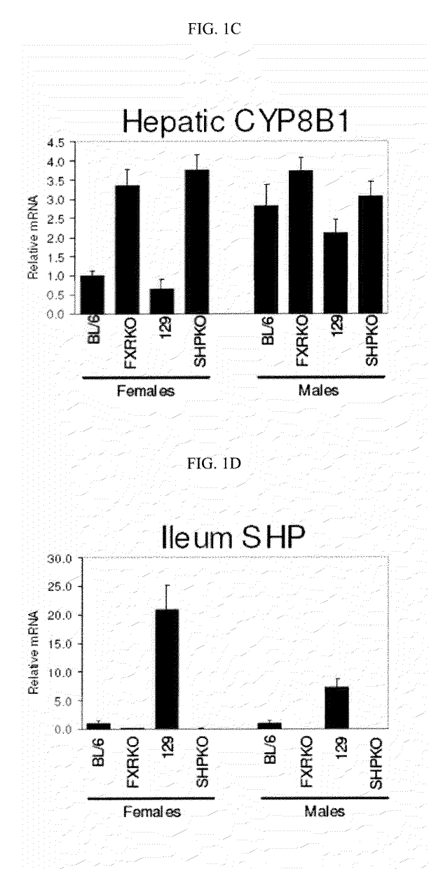

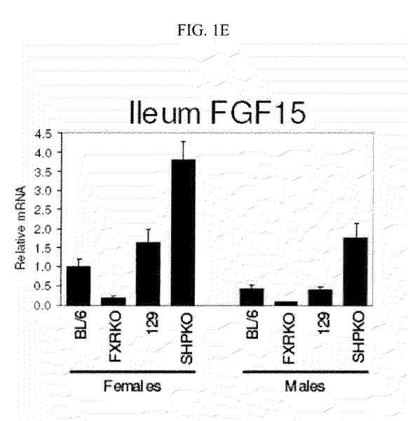

The nuclear hormone receptors FXR and SHP are critical to the control of bile acid synthesis. To determine the effects of loss of SHP on lipid metabolism, SHP knockout mice (SHPKO, SHP−/−) were created by generating mice containing a floxed SHP exon 1 followed by crossing them to mice containing a protamine CRE construct to generate a complete SHPKO. Total RNA was prepared from the livers and ileums of female and male mice between 8 and 10 weeks of age fed a standard chow diet. mRNA levels were quantified by real-time PCR, with expression levels normalized for GAPDH. The mean expression level in female C57BI/6 mice was defined as 1.0 for each gene. Expression of SHP was undetectable in the livers or ileums of these mice (FIGS. 1A and 1D, respectively). Similarly to FXRKO (FXR−/−) mice, the SHP−/− mice had elevated hepatic mRNA levels of the bile acid synthetic genes CYP7A1 and CYP8B1 (FIGS. 1B and 1C, respectively). In the ileum, FGF15 expression is driven by bile acid activation of FXR. Thus in the FXR−/− mice, FGF15 expression was reduced in the ileum, while FGF15 expression was increased in the ileum of SHP−/− (FIG. 1E). These results indicate that the SHP−/− had the expected effects on induction of bile acids synthetic genes and bile acid responsive genes in the ileum, similar to previous results with other SHP−/− mice. Values shown in FIG. 1 are the mean +/−SE (n=6 per group).

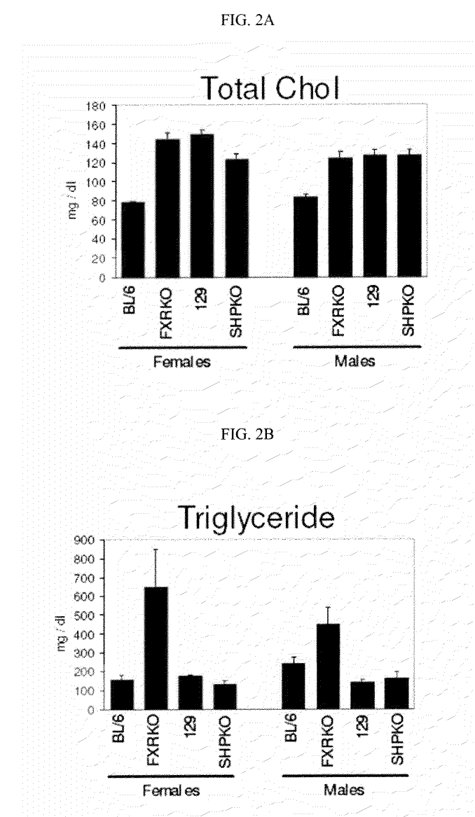

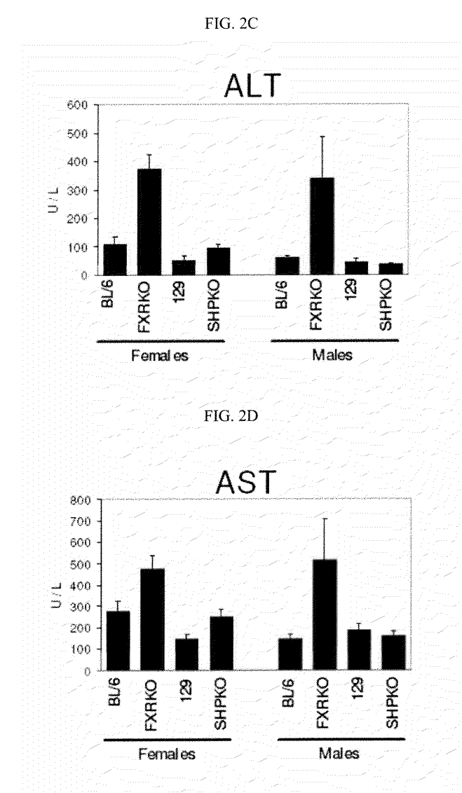

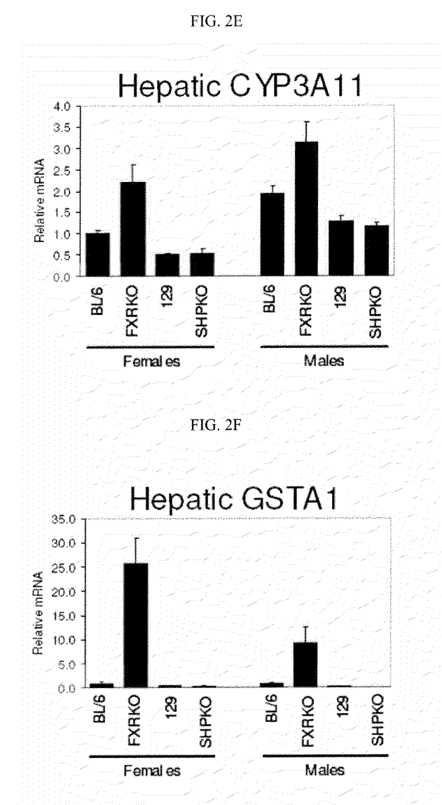

Lipid levels and inflammatory markers were examined in C57BI/6 WT mice, C57BI/6 FXR−/− mice, 129 WT mice, 129 SHP−/− mice. Total cholesterol and triglyceride levels along with ALT and AST levels in serum obtained from female and male mice between 8 and 10 weeks of age fed a standard chow diet were quantified using a Hitachi clinical chemistry analyzer. Although both FXR−/− and SHP−/− mice had similar effects on CYP7A1 and CYP8B1, FXR−/− mice had increased serum total cholesterol and triglyceride levels as compared to control mice, while SHP−/− mice had no change in lipid levels as compared to control mice (FIGS. 2A and 2B). Hepatic markers of inflammation including serum ALT and AST levels were elevated in the FXR−/− mice, but not in the SHP−/− mice (FIGS. 2C and 2D). Finally, expression of PXR regulated genes such as CYP3A11 and GSTA1 were increased in the FXR−/− mice but not in the SHP−/− mice (FIGS. 2E and 2F). The explanation for these changes in the FXR−/− mice has been proposed to be due to the proinflammatory effects of an expanded bile acid pool due to elevation of CYP7A1 and CYP8B1 expression in the FXR−/− mice. Since the SHP−/− mice had the same degree of elevation of CYP7A1 and CYP8B1, these results suggest that the loss of SHP is providing an offsetting beneficial activity. Hepatic mRNA levels were quantified by real-time PCR, with expression levels normalized for GAPDH. The mean expression level in female C57BI/6 mice was defined as 1.0 for each gene. Values shown are the mean +/−SE (n=6 per group).

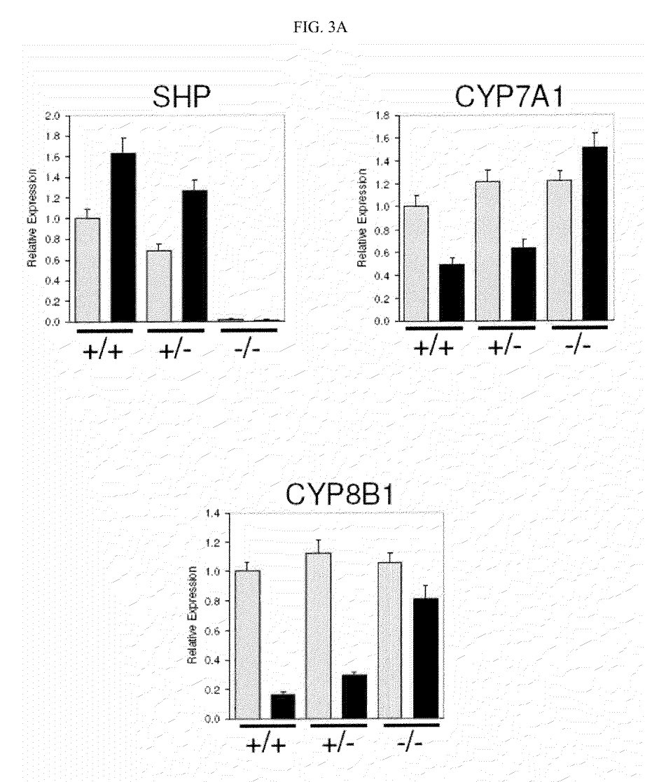

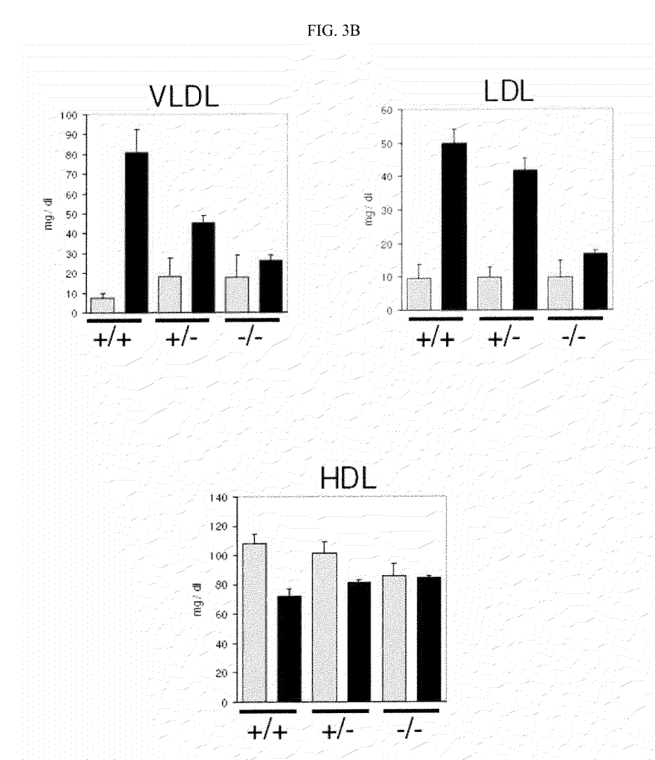

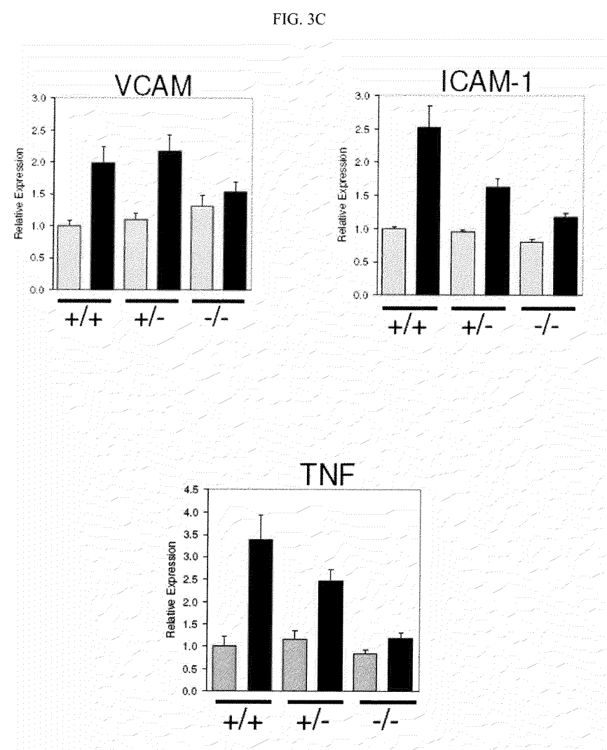

To further delineate the role of SHP in regulating serum lipids, SHP+/+, SHP+/−, and SHP−/− mice were generated by breeding of SHP+/− mice and fed a diet containing 2% cholesterol and 0.5% cholic acid (chol/CA) for 5 weeks. At 8 to 10 weeks of age, the mice were either maintained on a chow diet (FIG. 3, grey bars) or switched to a diet supplemented with 2% cholesterol and 0.5% cholic acid (FIG. 3, black bars). After 5 weeks of feeding, serum lipid levels and mRNA expression was determined. Hepatic SHP, CYP7A1, and CYP8B1 expression were quantified by real-time PCR with normalization for GAPDH expression. The expression level in SHP+/+ mice fed a chow diet was defined as 1.0. All values are the mean +/− SE (n=12 to 23 mice per group) (FIG. 3A). Serum total cholesterol levels were determined using a Hitachi 912 clinical chemistry analyzer. FPLC was performed to determine the cholesterol distribution in VLDL, LDL and HDL (FIG. 3B). Basal levels of hepatic SHP mRNA was partially reduced in the SHP+/− mice (FIG. 3A). In both the SHP+/+ and SHP+/− mice, the chol/CA diet increased SHP mRNA levels to a similar extent. Repression of CYP7A1 and CYP8B1 occurred to similar magnitude in both the SHP+/+ and SHP+/− mice, but was completely absent in the SHP−/− mice. The serum levels of VLDL and LDL were strongly increased in the SHP+/+ mice fed the chol/CA diet (FIG. 3B). These effects were accompanied by a decrease in the HDL cholesterol levels in these mice. In mice consuming a control chow diet, the levels of VLDL and LDL cholesterol were very similar in SHP+/+ and SHP−/− mice, and there was a small decrease in HDL cholesterol in the SHP−/− mice. The SHP−/− mice were highly resistant to the chol/CA diet elevations of VLDL and LDL cholesterol, as well as the decrease in HDL cholesterol, suggesting that SHP−/− mice were protected from diet-induced hypercholesterolemia. The SHP+/− generally had the same response as the SHP+/+ mice, although the magnitude of the VLDL cholesterol increase was attenuated in the SHP+/− mice.

Diets containing cholic acid are known to induce hepatic inflammation. In agreement with this, the expression of several inflammatory marker genes including VCAM, ICAM-1 and TNFα was observed to be increased in the livers of the SHP+/+ mice (FIG. 3C). Hepatic expression of vascular cell adhesion molecule (VCAM), intracellular adhesion molecule-1 (ICAM) and tumor necrosis factor α (TNFα) were determined by real-time PCR (FIG. 3C). The basal level of expression of these genes was not altered in the SHP−/− mice, and there was no induction of these genes in the SHP−/− mice by the chol/CA diet. In the SHP+/− mice, the chol/CA diet induced VCAM expression to the same magnitude as seen in the SHP+/+ mice. In contrast, the induction of genes such as ICAM-1 and TNFα was partially reduced in the SHP+/− mice. These results suggest that various inflammatory genes may have differing sensitivity to the loss of SHP.

The effects of some nuclear receptors such as FXR and PPARα on lipid metabolism have been shown to differ between male and female mice. To confirm that the protective effects of the loss of SHP occurred in both sexes, male and female SHP+/+ and SHP−/− mice were fed a chow or chol/CA (2% cholesterol, 0.5% cholic acid) diet for 5 weeks and 10 weeks. Both sexes showed a similar protection from diet-induced dyslipidemia (Table 1). Further, this protection was maintained when the mice were fed the chol/CA diet for 10 weeks, indicating a sustained protection from dietary dyslipidemia. Serum total cholesterol levels were determined using a Hitachi 912 clinical chemistry analyzer. FPLC was performed to determine the cholesterol distribution in VLDL, LDL and HDL.

TABLE 1
Lipoprotein Analysis of WT and SHPKO Mice

| VLDL | | LDL | | HDL | |
|---|---|---|---|---|---|
| Chow | Chol/CA | Chow | Chol/CA | Chow | Chol/CA |

5-week

| | | | | | | |
|---|---|---|---|---|---|---|
| WT-Male | 4 ± 0 | 87 ± 9 | 5 ± 1 | 71 ± 6 | 127 ± 2 | 96 ± 5 |
| SHPKO-Male | 5 ± 1 | 24 ± 6 | 5 ± 0 | 24 ± 4 | 112 ± 5 | 85 ± 7 |
| WT-female | 21 ± 8 | 118 ± 11 | 23 ± 9 | 44 ± 3 | 91 ± 16 | 73 ± 8 |
| SHPKO-female | 33 ± 14 | 51 ± 11 | 21 ± 7 | 20 ± 2 | 70 ± 13 | 58 ± 12 |

10-week

| | | | | | | |
|---|---|---|---|---|---|---|
| WT-Male | 10 ± 3 | 125 ± 25 | 21 ± 15 | 61 ± 13 | 114 ± 17 | 63 ± 24 |
| SHPKO-Male | 13 ± 5 | 25 ± 3 | 9 ± 3 | 24 ± 4 | 85 ± 13 | 84 ± 9 |
| WT-female | 9 ± 0 | 130 ± 11 | 8 ± 1 | 46 ± 7 | 100 ± 7 | 66 ± 13 |
| SHPKO-female | 69 ± 24 | 68 ± 20 | 6 ± 1 | 13 ± 3 | 56 ± 22 | 76 ± 18 |

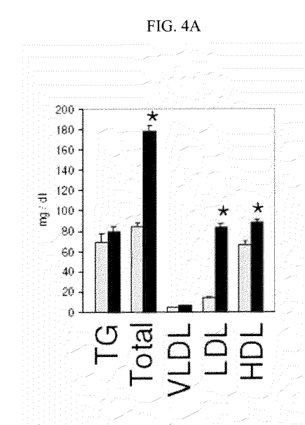

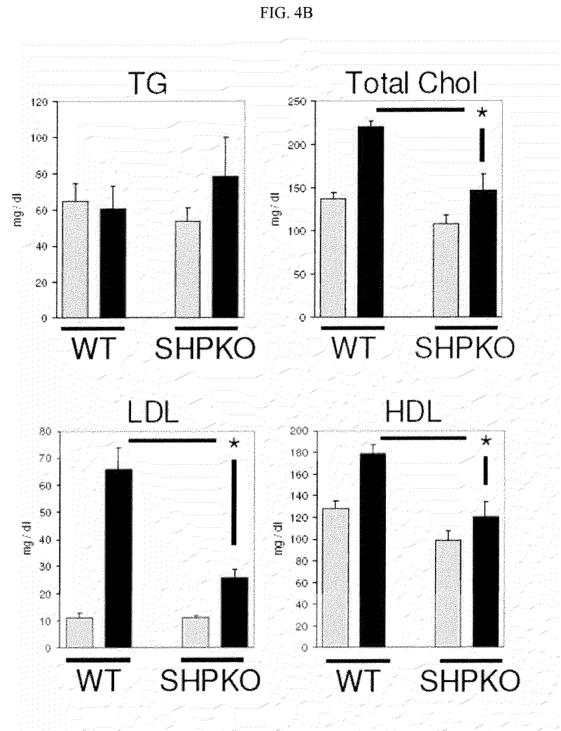

Because the chol/CA diet contains supraphysiological levels of cholic acid and can activate expression of SHP, we determined whether SHP−/− mice would also be protected by dyslipidemia resulting from hypothyroidism. C57BI/6 mice at 8 to 10 weeks of age were maintained on a standard chow diet (FIG. 4A, grey bars) or switched to an iodine-deficient diet supplemented with propylthiouracil (FIG. 4A, black bars) for three weeks. Serum total triglyceride and cholesterol levels were determined using a Hitachi clinical chemistry analyzer. VLDL, LDL and HDL cholesterol levels were determined by FPLC. *p<0.01 for diet induced change in lipoprotein levels (n=6 males and 6 females per group). In this model, mice fed a diet that produces hypothyroidism have an increase in total cholesterol, primarily due to a large increase in LDL cholesterol accompanied by a small increase in HDL cholesterol (FIG. 4A).

SHP+/+ and SHP−/− mice at 8 to 10 weeks of age were maintained on a standard chow diet (FIG. 4B, grey bars) or switched to an iodine-deficient diet supplemented with propylthiouracil (FIG. 4B, black bars) for three weeks. Serum total triglyceride, total cholesterol, LDL cholesterol, and HDL cholesterol levels were determined using a Hitachi clinical chemistry analyzer. *p<0.01 for comparison between SHP+/+ and SHP−/− mice (n=4 males and 4 females per group). Thyroid hormone status has no effect on SHP expression. Hypothyroid SHP−/− mice had a significantly smaller increase in total, LDL and HDL cholesterol (FIG. 4B), confirming the protective effects of the loss of SHP is independent of diets containing cholic acid.

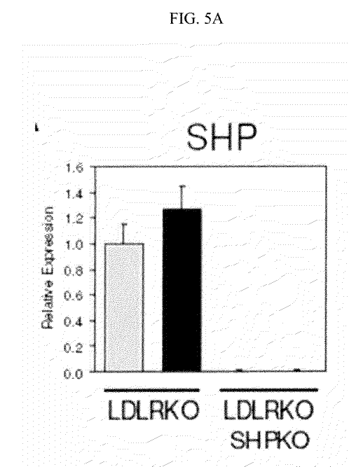

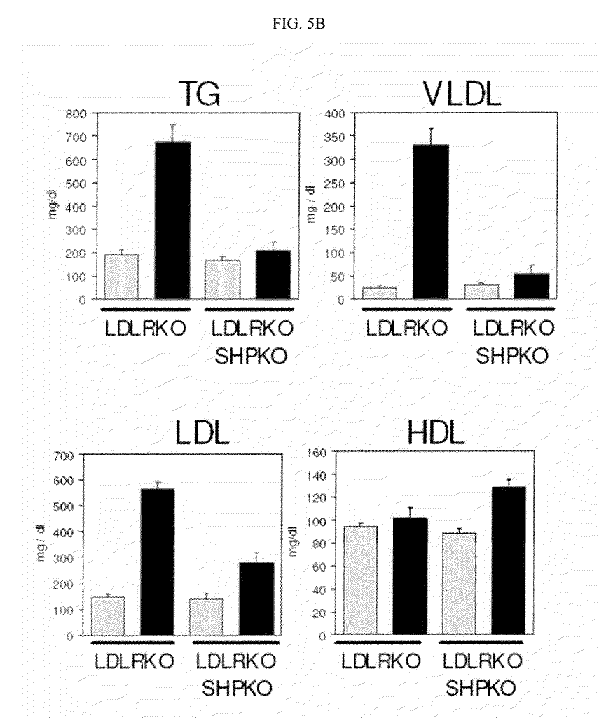

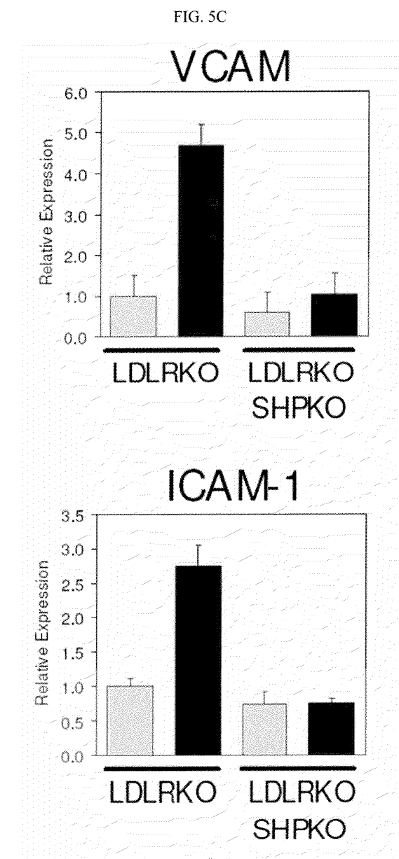

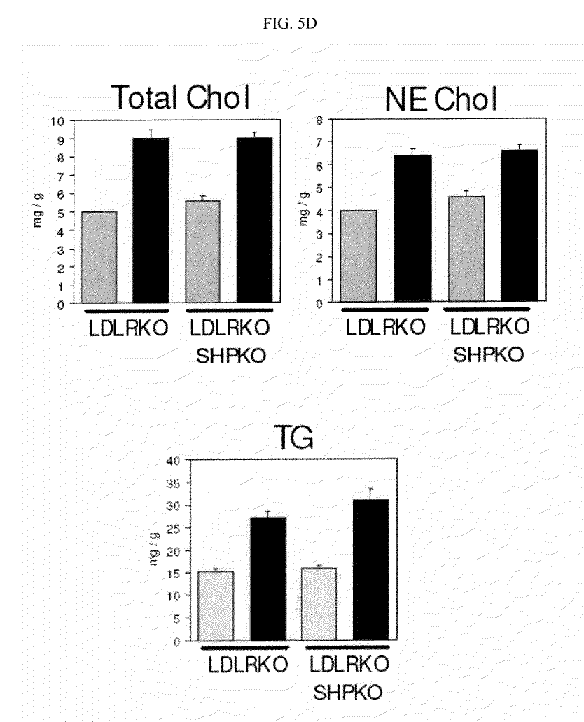

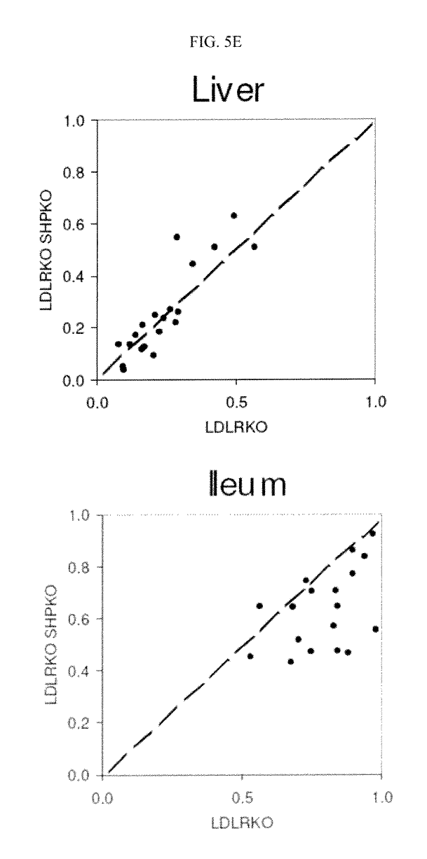

To examine whether the loss of SHP is protective in a mouse atherosclerosis model, LDLR−/− mice were crossed with SHP−/− mice to generate LDLR+/−SHP+/− mice, which were then interbred to generate LDLR−/− SHP−/− mice. At 8 to 10 weeks of age, LDLR−/− and LDLR−/−SHP−/− male mice (n=5 per group) were either maintained on a chow diet or placed on a Western diet for 7 days. SHP−/− mice were crossed with LDLR−/− to generate LDLR−/−SHP−/− mice. Hepatic SHP mRNA was quantified by real-time PCR, with values normalized for GAPDH expression. Expression in LDLR−/− mice fed a chow diet was defined as 1.0 (FIG. 5A).

When the LDLR−/− mice were fed a Western diet for 7 days, serum triglyceride (TG), VLDL cholesterol, and LDL cholesterol were greatly increased (FIG. 5B). Serum total cholesterol and triglyceride levels were determined using a Hitachi 912 clinical chemistry analyzer. VLDL, LDL and HDL cholesterol levels (mg/dl) were determined by FPLC.

In contrast, the LDLR−/−SHP−/− mice consuming a Western diet showed no increase in TG levels, and had greatly reduced elevations in VLDL and LDL cholesterol levels. The Western diet had no effect on HDL cholesterol levels in the LDLR−/− mice, while HDL cholesterol levels were increased by the Western diet in the LDLR−/−SHP−/− mice. The Western diet also resulted in increased hepatic expression of hepatic inflammatory marker genes including VCAM, ICAM-1 and TNFα in the LDLR−/− mice (FIG. 5C). Hepatic VCAM, ICAM-1, and TNFα expression was determined by real-time PCR. Again the loss of SHP expression in the LDLR−/−SHP−/− mice resulted in a near complete block in the induction of these genes.

While not wishing to be bound by theory, a potential explanation for the protection of the LDLR−/− SHP−/− mice would be if cholesterol were simply not taken up from the diet. However, hepatic content of total and nonesterified cholesterol as well as triglyceride was increased to the same extent by Western diet feeding in the LDLR−/− and LDLR−/− SHP−/− mice (hepatic total cholesterol, nonesterified cholesterol, and triglyceride content (mg/g) was determined as previously described; FIG. 5D). To confirm these results, mRNA levels for a panel of 20 genes involved in cholesterol metabolism were quantified (Table 2). There was a very high correlation between the magnitude of repression of these genes in the LDLR−/− and LDLR−/−SHP−/− mice (FIG. 5E), consistent with equivalent increases in hepatic cholesterol content in the LDLR−/− and LDLR−/−SHP−/− mice. The expression of the panel of 20 cholesterol regulated genes was determine by GeneChip as described in Table 2. The fold repression by the Western diet in the LDLR−/−SHP−/− mice was plotted against the fold repression by the Western diet in the LDLR−/− mice. Although cholesterol content of the ileum could not be measured directly due to diet remaining within the tissue sample, repression of the set of 20 cholesterol metabolism genes was used to determine cholesterol content of the ileum. For all genes, repression was either the same or greater in the LDLR−/−SHP−/− mice as compared to the LDRL−/− mice, suggesting that cholesterol levels in the enterocytes of the LDLR−/−SKHP−/− mice were at least the same or greater than the LDLR−/− mice.

TABLE 2

| | | | | | LDLRKO | | LDLRKO | | LDLRKO |
|---|---|---|---|---|---|---|---|---|---|
| | LDLRKO | | LDLRKO | | SHPKO | | SHPKO | | SHPKO |
| | Chow | | Western Diet | | Chow | | Western Diet | | Fold |
| Gene | | SE | | SE | | SE | | SE | Change |
| LIVER: | | | | | | | | | |
| Acetoacetyl-CoA synthase | 1.00 | 0.16 | 0.29 | 0.03 | 0.86 | 0.20 | 0.47 | 0.10 | 0.55 |
| Acetyl-CoA-synthetase | 1.00 | 0.23 | 0.34 | 0.08 | 0.59 | 0.04 | 0.26 | 0.02 | 0.44 |
| ATP citrate lyase | 1.00 | 0.15 | 0.42 | 0.07 | 0.74 | 0.03 | 0.38 | 0.01 | 0.51 |
| 7-dehydrocholesterol reductase | 1.00 | 0.09 | 0.29 | 0.04 | 1.13 | 0.15 | 0.29 | 0.02 | 0.26 |
| Farnesyl diphosphate synthetase | 1.00 | 0.13 | 0.16 | 0.05 | 1.00 | 0.09 | 0.12 | 0.02 | 0.12 |
| Squalene synthase | 1.00 | 0.13 | 0.16 | 0.02 | 0.84 | 0.13 | 0.18 | 0.01 | 0.21 |
| 3-hydroxy-3-methylglutaryl-CoA | 1.00 | 0.15 | 0.17 | 0.03 | 0.90 | 0.22 | 0.11 | 0.02 | 0.13 |
| synthase 1 | | | | | | | | | |
| 3-hydroxy-3-methylglutaryl-CoA | 1.00 | 0.16 | 0.14 | 0.03 | 0.71 | 0.16 | 0.12 | 0.02 | 0.17 |
| reductase | | | | | | | | | |
| Isopentenyl-diphosphate delta | 1.00 | 0.16 | 0.10 | 0.01 | 0.92 | 0.19 | 0.03 | 0.01 | 0.04 |
| isomerase | | | | | | | | | |
| Lanosterol 14 a-demethylase | 1.00 | 0.11 | 0.20 | 0.03 | 0.65 | 0.11 | 0.06 | 0.02 | 0.09 |
| Mevalonate (diphospho) | 1.00 | 0.09 | 0.12 | 0.04 | 0.86 | 0.16 | 0.12 | 0.01 | 0.14 |
| decarboxylase | | | | | | | | | |
| NAD(P) dependent steroid | 1.00 | 0.13 | 0.23 | 0.05 | 0.99 | 0.14 | 0.18 | 0.02 | 0.18 |
| dehydrogenase-like | | | | | | | | | |
| Phosphomevalonate kinase | 1.00 | 0.16 | 0.24 | 0.05 | 0.85 | 0.08 | 0.20 | 0.02 | 0.24 |
| Lanosterol synthase | 1.00 | 0.14 | 0.21 | 0.05 | 1.19 | 0.19 | 0.30 | 0.07 | 0.25 |
| Squalene epoxidase | 1.00 | 0.12 | 0.09 | 0.04 | 0.96 | 0.15 | 0.05 | 0.01 | 0.05 |
| Sterol-C4-methyl oxidase-like | 1.00 | 0.13 | 0.08 | 0.01 | 1.15 | 0.20 | 0.16 | 0.02 | 0.14 |
| Sterol-C5-desaturase | 1.00 | 0.11 | 0.49 | 0.05 | 0.78 | 0.04 | 0.49 | 0.03 | 0.63 |
| Insulin induced gene 1 | 1.00 | 0.05 | 0.26 | 0.04 | 0.81 | 0.08 | 0.22 | 0.01 | 0.27 |
| Proprotein convertase | 1.00 | 0.17 | 0.28 | 0.04 | 0.92 | 0.08 | 0.20 | 0.06 | 0.22 |
| subtilisin/kexin type 9 | | | | | | | | | |
| Sterol regulatory element binding | 1.00 | 0.08 | 0.57 | 0.03 | 0.87 | 0.09 | 0.44 | 0.04 | 0.51 |
| factor 2 | | | | | | | | | |
| ILEUM: | | | | | | | | | |
| Acetoacetyl-CoA synthase | 1.00 | 0.01 | 0.47 | 0.03 | 0.75 | 0.03 | 0.53 | 0.05 | 0.70 |
| Acetyl-CoA-synthetase | 1.00 | 0.07 | 0.84 | 0.09 | 0.94 | 0.14 | 0.78 | 0.06 | 0.84 |
| ATP citrate lyase | 1.00 | 0.06 | 0.79 | 0.04 | 0.97 | 0.10 | 0.89 | 0.07 | 0.92 |
| 7-dehydrocholesterol reductase | 1.00 | 0.04 | 0.82 | 0.03 | 1.29 | 0.06 | 0.78 | 0.06 | 0.60 |
| Farnesyl diphosphate synthetase | 1.00 | 0.06 | 0.46 | 0.04 | 0.70 | 0.04 | 0.36 | 0.03 | 0.52 |
| Squalene synthase | 1.00 | 0.05 | 0.41 | 0.02 | 0.84 | 0.04 | 0.40 | 0.03 | 0.47 |
| 3-hydroxy-3-methylglutaryl-CoA | 1.00 | 0.04 | 0.54 | 0.02 | 0.83 | 0.02 | 0.47 | 0.03 | 0.57 |
| synthase 1 | | | | | | | | | |
| 3-hydroxy-3-methylglutaryl-CoA | 1.00 | 0.03 | 0.59 | 0.03 | 0.84 | 0.12 | 0.54 | 0.07 | 0.65 |
| reductase | | | | | | | | | |
| Isopentenyl-diphosphate delta | 1.00 | 0.12 | 0.41 | 0.07 | 0.98 | 0.08 | 0.54 | 0.04 | 0.55 |
| isomerase | | | | | | | | | |
| Lanosterol 14 a-demethylase | 1.00 | 0.08 | 0.40 | 0.04 | 0.68 | 0.06 | 0.29 | 0.04 | 0.43 |
| Mevalonate (diphospho) | 1.00 | 0.07 | 0.53 | 0.06 | 0.68 | 0.02 | 0.44 | 0.03 | 0.64 |
| decarboxylase | | | | | | | | | |
| NAD(P) dependent steroid | 1.00 | 0.06 | 0.59 | 0.03 | 0.84 | 0.03 | 0.59 | 0.04 | 0.71 |
| dehydrogenase-like | | | | | | | | | |
| Phosphomevalonate kinase | 1.00 | 0.06 | 0.55 | 0.05 | 0.88 | 0.12 | 0.41 | 0.04 | 0.47 |
| Lanosterol synthase | 1.00 | 0.11 | 0.60 | 0.02 | 0.73 | 0.09 | 0.54 | 0.03 | 0.74 |
| Squalene epoxidase | 1.00 | 0.05 | 0.43 | 0.03 | 0.75 | 0.07 | 0.35 | 0.04 | 0.47 |
| Sterol-C4-methyl oxidase-like | 1.00 | 0.10 | 0.27 | 0.01 | 0.53 | 0.08 | 0.24 | 0.01 | 0.45 |
| Sterol-C5-desaturase | 1.00 | 0.07 | 0.85 | 0.03 | 0.90 | 0.08 | 0.77 | 0.02 | 0.86 |
| Insulin induced gene 1 | 1.00 | 0.08 | 0.57 | 0.02 | 1.15 | 0.08 | 0.64 | 0.03 | 0.56 |
| Proprotein convertase | 1.00 | 0.06 | 0.49 | 0.07 | 0.90 | 0.22 | 0.69 | 0.08 | 0.77 |
| subtilisin/kexin type 9 | | | | | | | | | |
| Sterol regulatory element binding | 1.00 | 0.13 | 0.60 | 0.13 | 0.56 | 0.06 | 0.36 | 0.04 | 0.65 |
| factor 2 | | | | | | | | | |

Table 2 supports the conclusion that a Western diet represses cholesterol metabolism genes similarly in the livers and ileums of LDLR−/−SHP−/− and LDLR−/− mice. Total RNA prepared from the livers and ileums of LDLR−/− and LDLR−/−SHP−/− mice were assayed by MOE430 v2.0 GeneChips. RNA from each individual animal was hybridized separately. The mean expression in LDLR−/− mice fed a chow diet was defined as 1.0 for each gene. Values are the mean expression level +/−SE.

A potential explanation for the decreased serum cholesterol levels in the context of increased hepatic cholesterol levels is for increase cholesterol elimination. In the LDLR−/− mice, Western diet feeding did not change the expression of CYP7A1 (FIG. 5F). This result may be due to simultaneous activation of CYP7A1 by increased cholesterol levels (mediated by LXR) and repression of CYP7A1 by increased bile acid levels (mediated by the FXR/SHP pathway). In agreement with this hypothesis, the Western diet repressed CYP8B1 expression in the LDLR−/− mice, and induced expression of FGF15 in the ileum, both physiological markers for an expanded bile acid pool size. In contrast, the LDLR−/−SHP−/− mice fed control chow had elevated expression of CYP7A1, and elevated expression of FGF15 in the ileum. Western diet feeding paradoxically induced expression of CYP7A1 in these mice. This would be consistent with these mice having retained LXR activation of CYP7A1 expression but no countervailing FXR/SHP mediated repression of CYP7A1 expression. Expression of CYP7A1 and CYP8B1 in the liver and expression of FGF15 in the ileum were determined by real-time PCR.

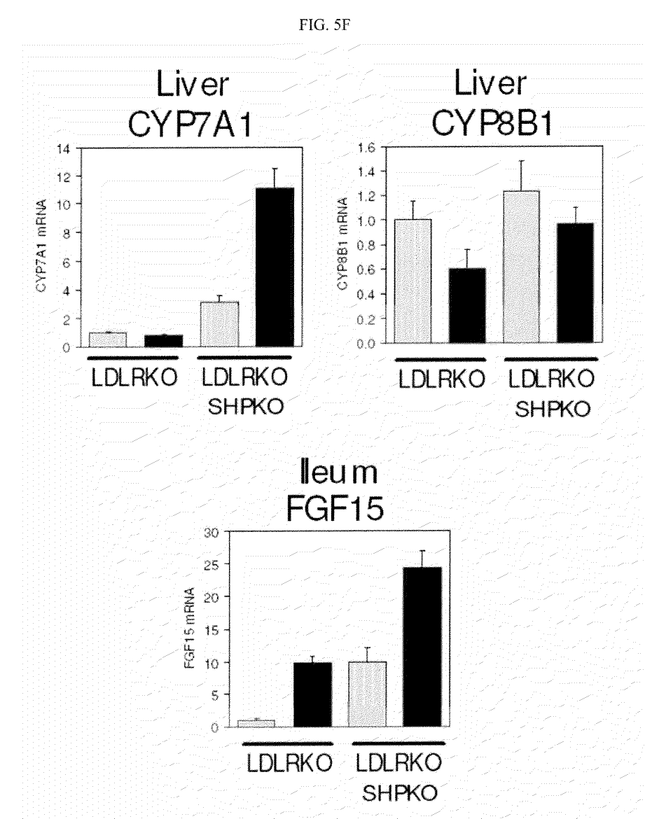

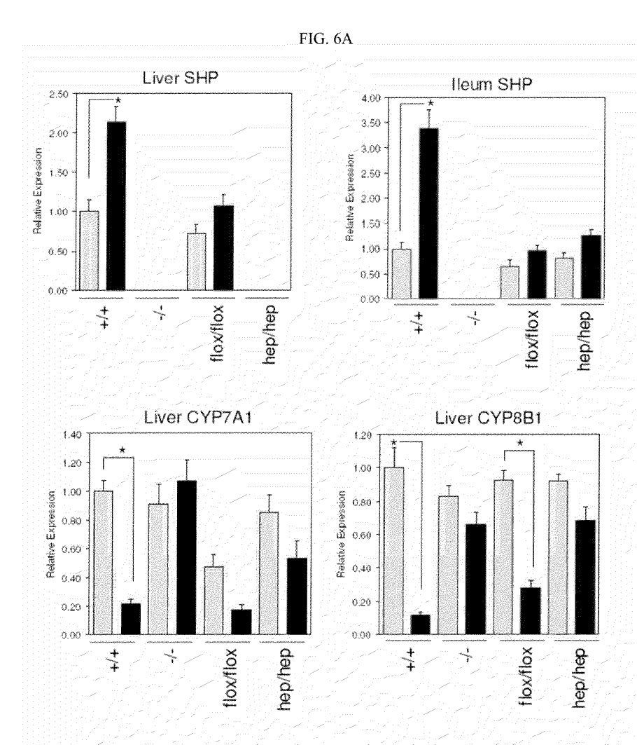

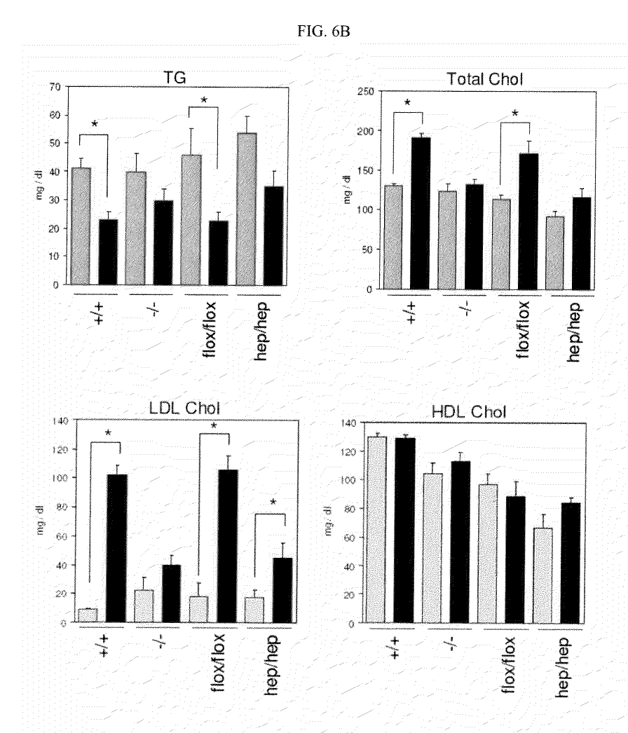

Although SHP is expressed in multiple cell types throughout the body, the above results suggest that the loss of SHP expression specifically within the hepatocyte is responsible for the protection against dyslipidemia. To confirm the conclusion that selective loss of SHP expression in hepatocytes protects against diet-induced dyslipidemia, mice containing the floxed SHP gene exon 1 (SHPflox/flox) were crossed with mice expressing Cre under the control of the albumin promoter to generate mice selectively deficient in SHP in the hepatocyte (SHPhep/hep, FIG. 6A). SHP+/+, SHP−/−, SHPflox/flox, and SHPhep/hep mice at 8 to 10 weeks of age were either maintained on a chow diet (FIG. 6, grey bars) or switched to a diet supplemented with 2% cholesterol and 0.5% cholic acid (FIG. 6, black bars) for 5 weeks. Gene expression in the liver and ileum was quantified by real-time PCR. All values were normalized for GAPDH expression, with expression in SHP+/+ mice on a chow diet defined as 1.0 for each gene. *p<0.01 (n³ 12 for each group) (FIG. 6A). Induction of SHP by the chol/CA diet was reduced in the liver and ileum of the SHPflox/flox mice and the ileum of the SHPhep/hep mice, possibly due either to an effect of the flanking lox elements or the mixed 129; C57BI/6 background of these mice on SHP gene regulation. However, repression of CYP8B1 expression by the chol/CA diet was clearly abolished in both the SHP−/− and the SHPhep/hep mice, indicating that hepatic SHP expression is the critical factor in CYP8B1 repression. Interestingly, repression of hepatic CYP7A1 expression was similar in the SHPflox/flox control mice and the SHPhep/hep mice (although both strains had less repression than seen in the SHP+/+ mice). These effects may be due to the diminished induction of SHP, but may also suggest that SHP expression in other cell types is of critical importance for CYP7A1 regulation.

The chol/CA diet resulted in a strong decrease in serum TG levels in SHP+/+ and SHPflox/flox mice (FIG. 6B). Serum total triglyceride, total cholesterol, LDL cholesterol, and HDL cholesterol were determine using a Hitachi 912 clinical chemistry analyzer. *p<0.01 (n³12 for each group). The mechanisms for FXR regulation of TG levels are diverse, and have been reported to include both a SHP-mediated pathway via repression of SREBP-1 and SHP-independent pathways such as regulation of apoC2 and apoC3 expression. In agreement with these models, TG levels were still reduced by the chol/CA diet in both the SHP−/− and SHPhep/hep mice, although the magnitude of reduction was less and no longer statistically significant (p<0.01). The chol/CA diet inductions of total cholesterol and LDL cholesterol that occurred in the SHP+/+ and SHPflox/flox mice were strongly reduced in the SHP−/− and SHPhep/hep mice. Further, the reduction seen in the SHP−/− and SHPhep/hep mice was indistinguishable. These results indicate that it is SHP within the hepatocyte that functions as a critical regulator of serum cholesterol levels.

What is claimed is:

1. A method for reducing the level of very low density lipoprotein (“VLDL”) or low density lipoprotein (“LDL”) in serum of a subject, the method comprising inhibiting the activity of small heterodimer partner (“SHP”) in the subject.

2. The method according to claim 1 wherein the subject suffers from familial hypercholesterolemia.

3. The method according to claim 1 wherein the subject suffers from hypothyroidism.

4. The method according to claim 1 wherein the subject has diet-induced dyslipidemia.

5. The method according to claim 1 wherein the inhibition of SHP activity is followed by an increase in expression of FGF 15 in the ileum of the subject.

6. The method according to claim 1 wherein the inhibition of SHP activity is followed by a decrease in the level of serum triglycerides or fatty acids in the subject.

7. The method according to claim 1, wherein the patient is a human.

8. The method according to claim 1, wherein the step of inhibiting the activity of SHP comprises administering a therapeutically effective amount of a polynucleotide to the subject.

9. The method of claim 8 wherein the polynucleotide is an antisense polynucleotide.

10. The method of claim 9, wherein the polynucleotide comprises at least 10 consecutive nucleic acids that are capable of hybridizing to a portion of SEQ ID NO:1.

11. The method according to claim 10 wherein the polynucleotide is capable of causing a reduction in the amount of SHP produced in the liver.

12. The method of claim 11, wherein the reduction comprises selective degradation of the SHP messenger RNA produced in the liver.

13. The method of claim 11, wherein the reduction comprises the inhibition of translation of the SHP messenger RNA in the liver.

14. The method of claim 8, wherein the polynucleotide is an siRNA molecule.

15. The method of claim 14, wherein the siRNA molecule comprises at least 10 consecutive nucleic acids that are capable of hybridizing to a portion of SEQ ID NO:1.

16. The method of claim 8, wherein the polynucleotide is an shRNA molecule.

17. The method of claim 16, wherein the shRNA molecule comprises at least about 30 nucleic acids that are capable of hybridizing to a portion of SEQ ID NO:1.

18. The method according to claim 1, wherein the step of inhibiting the activity of SHP comprises administering a therapeutically effective amount of a compound to the subject, wherein the compound that is delivered to the subject is capable of being metabolized in the liver into an inhibitor of SHP.

19. The method according to claim 1, wherein the step of inhibiting the activity of SHP comprises administering a therapeutically effective amount of a compound to the subject, wherein the compound is capable of inhibiting SHP activity or inhibiting SHP production.

20. The method according to claim 1, wherein the step of inhibiting the activity of SHP comprises administering a therapeutically effective amount of a compound to the subject, wherein the compound is capable of being metabolized into a farnesoid X receptor (“FXR”) antagonist in the liver.

21. The method according to claim 1, wherein the step of inhibiting the activity of SHP comprises administering a therapeutically effective amount of a compound to the subject, wherein the compound is capable of being metabolized into an estrogen receptor (“ER”) antagonist in the liver.

22. A composition useful in the treatment of hypercholesterolemia, the composition comprising an inhibitor of SHP activity in a pharmaceutically acceptable excipient.

23. The composition according to claim 22 wherein the inhibitor of SHP activity is selected from the group consisting of an isolated antisense polynucleotide, an siRNA polynucleotide, an shRNA, a compound that blocks SHP binding to its endogenous target, a compound that inhibits an upstream effector of SHP, and a compound that is metabolized in the liver of a subject to form a second compound that inhibits an upstream effector of SHP.

24. The composition of claim 23 wherein the upstream effector is a farnesoid X receptor (“FXR”) or an estrogen receptor (“ER”).

25. The composition of claim 23 wherein the compound that blocks SHP binding to its endogenous target is selected from the group consisting of SHP-specific antibody, SHP-specific antibody fragment, histone 3, histone 3 fragment, histone 3 analog, HDAC-1, HDAC-1 fragment, HDAC-1 analog, G9a, G9a fragment, and G9a analog.

26. The composition of claim 23 wherein the isolated antisense polynucleotide comprises at least 10 nucleotides that are capable of hybridizing to a portion of SEQ ID NO:1.

27. The composition of claim 23 wherein the siRNA comprises at least 10 nucleotides that are capable of hybridizing to a portion of SEQ ID NO:1.

28. The composition of claim 23 wherein the siRNA comprises at least about 30 nucleotides that are capable of hybridizing to a portion of SEQ ID NO:1.
